(19) United States
(12) Patent Application Publication
(10) Pub. No.: US 2021/0087548 A1
(43) Pub. Date: Mar. 25, 2021

(54) Compositions and Methods for Inducing Protein Function

(71) Applicant: The Board of Trustees of the Leland Stanford Junior University, Stanford, CA (US)

(72) Inventors: Michael Z. Lin, Stanford, CA (US); Conor Jacobs, Stanford, CA (US); Ryan Badiee, Redwood City, CA (US)

(21) Appl. No.: 16/634,040

(22) PCT Filed: Jul. 24, 2018
(86) PCT No.: PCT/US2018/043377

§ 371 Date: Jan. 24, 2020

Related U.S. Application Data
(60) Provisional application No. 62/536,307, filed on Jul. 24, 2017.

(51) Int. Cl. C12N 9/96; C12N 9/50; C12N 7/00; C12N 15/90; C12N 9/22

(52) U.S. Cl. CPC C12N 9/96; C12Y 304/21098; C12N 9/506; C12N 7/00; C12N 2800/80; C12N 9/22; C07K 2319/50; C12N 2770/24222; C12N 15/907

(57) ABSTRACT

Provided herein are compositions and methods for inducing protein function. For example, in some embodiments, provided herein are compositions and methods for pharmacological induction of protein function.

### CROSS-REFERENCE TO RELATED APPLICATIONS

The present invention claims the priority benefit of U.S. Provisional Patent Application 62/536,307 filed Jul. 24, 2017, the contents of which is incorporated by reference in its entirety.

This invention was made with Government support under contract 5R01GM098734 awarded by the National Institutes of Health. The Government has certain rights in the invention.

### FIELD

Provided herein are compositions and methods for inducing protein function. For example, in some embodiments, provided herein are compositions and methods for pharmacological induction of protein function.

### BACKGROUND

Generalizable methods for pharmacological induction of protein function are highly useful for gene- or cell-based therapies, as well as for studying temporal requirements for proteins in living contexts. Existing methods include attaching a protein of interest to a destabilization domain whose stability can be enhanced by drug binding, and fusing complementary fragments of a protein to domains whose heterodimerization can be induced by drug binding (Rakhit, R., et al., Chem. Biol. 21, 1238-1252 (2014)). However, the first type of method is often limited by leaky protein expression in the absence of drug (Armstrong, C. M. & Goldberg, D. E. Nat Methods 4, 1007-1009 (2007); Liu, Y. C. & Singh, U. Int J Parasitol 44, 729-735 (2014)), while the second method can exhibit basal reconstitution of the protein in the absence of drug (Gray, D. C., et al., Cell 142, 637-646 (2010); Massoud, T. F., et al., Zetsche, B., et al., Nat Biotechnol 33, 139-142 (2015)) and/or poor reconstitution in the presence of drug (Massoud et al., supra; Zetsche et al., supra). Furthermore, fragment complementation requires the expression of two polypeptides for each activity to be regulated, making the simultaneous regulation of multiple activities cumbersome.

What are needed are new approaches for pharmacological induction of a protein function of interest that entails only the expression of a single polypeptide and are robust, generalizable, and multiplexable.

### SUMMARY

Provided herein are compositions and methods for inducing protein function. For example, in some embodiments, provided herein are compositions and methods for pharmacological induction of protein function.

Existing methods to induce protein function with drugs require the expression of multiple genes, are difficult to generalize, or do not perform robustly. Provided herein is a generalizable method for creating proteins with drug-inducible activity that is robust and multiplexible.

Accordingly, in some embodiments, provided herein is a composition, comprising: a fusion protein comprising a) a first polypeptide of interest; and b) a first protease and a substrate for the protease. In some embodiments, the protease and the substrate are inserted between multiple domains of the polypeptide. In some embodiments, the protease and the substrate are inserted within a domain of the polypeptide of interest. In some embodiments, the protease and the substrate are inserted between two copies of the polypeptide of interest. In some embodiments, the composition comprises a second fusion protein comprising a second polypeptide of interest and a second protease, wherein the second protease is distinct from said first protease. In some embodiments, the first protease and the second protease are inhibited by different protease inhibitors. The present disclosure is not limited to particular proteases. In some embodiments, the first and second proteases are HCV NS3 proteases. In some embodiments, the protease comprises at least one mutation. For example, in some embodiments, the first HCV NS3 protease comprises V36M, T54A, and S122G mutations and the second HCV NS3 protease comprises F43L, Q80K, S122R, and D168Y mutations, although other mutations are specifically contemplated. In some embodiments, the HCV NS3 protease comprising V36M, T54A, and S122G mutations is resistant to telaprevir (TPV) and sensitive to asunaprevir (ASV) and the HCV NS3 protease comprising F43L, Q80K, S122R, and D168Y mutations is resistant to ASV and sensitive to TPV.

The present disclosure is not limited to particular polypeptides of interest. In some embodiments, the polypeptide of interest is, for example, a transcription factor, a nuclease enzyme, a protease enzyme, or a metabolic enzyme.

Further embodiments provide a nucleic acid encoding the fusion proteins described herein. In some embodiments, the nucleic acid is on a vector.

Yet other embodiments provide a cell comprising the nucleic acids or fusion proteins described herein. In some embodiments, the nucleic acid is on a chromosome of a cell. In some embodiments, the cell is in an organism (e.g., a microorganism, a non-human animal, or a human).

Still other embodiments provide a kit or system, comprising: a) a nucleic acid as described herein; and b) at least one protease inhibitor.

Certain embodiments provide a method of modulating the activity or function of a polypeptide of interest, comprising: contacting the cell with a protease inhibitor under conditions such that the polypeptide of interest is active.

Further embodiments are described herein.

### BRIEF DESCRIPTION OF THE DRAWINGS

FIG. 1 shows (A) Three exemplary architectures for deploying stabilizable protein linkages (StaPLs). A StaPL sequence, comprising a HCV NS3 protease domain and a cognate substrate sequence linked in cis, can be used to connect two protein domains in an artificial multidomain protein (left), to connect two portions of a natural protein domain (center), or to connect two halves of a tandem dimer for a given protein (right). (B) HCV NS3 protease inhibitors used in this study. The asterisk marks the site of covalent bond formation between TPV and the catalytic serine. (C) Dose-inhibition curves for NS3(ai), NS3(ti), and wild-type NS3 with ASV and TPV, quantified as suppression of SMASh tag self-cleavage from PSD95-SMASh variants transiently expressed in HEK293A cells. Error bars, ±standard error of the mean. (D) Models of NS3(ai) and NS3(ti) were created by mutagenesis of published X-ray structures of NS3-ASV and NS3-TPV co-crystals (PDB entries 4WF8 and 3SV6), followed by energy minimization. (E) In transiently transfected HeLa cells, expression of YFP from a YFP-SMASh(ai) construct is suppressed by ASV but not TPV, whereas expression of YFP from a YFP-SMASh(ti) construct is suppressed by TPV but not ASV. Live cells imaged 24 hrs after transfection. Scale bar, 20 um. (F) With ASV and TPV, SMASh tagging of PSD95 and Arc can be controlled independently and orthogonally in a transiently transfected HeLa cells. Cells coexpressed Arc and PSD95 with SMASh tags containing orthogonal NS3 variants for 24 hrs in indicated drug.

FIG. 2 shows orthogonal control of nuclear localization of tdYFP and tdRFP by conditional linkage of an NLS by StaPL(ai) and StaPL(ti) sequences. Orthogonal STaPL modules allow for independent, simultaneous control of nuclear localization for two fluorescent proteins (FPs). Top, schematics of constructs for small molecule control of nuclear localization. Tandem YFPs or RFPs were fused to a nuclear localization signal (NLS), with an intervening STaPL module and cleavage site governing preservation of the NLS. Bulkiness of tandem FPs ensure no passive diffusion occurs in absence of NLS. Bottom left, HEK293A cells were transiently transfected with the indicated constructs and incubated in the indicated drugs. For tdYFP-StaPL(ai)-NLS, concentrated nuclear YFP fluorescence is observed in ASV but signal is nuclear excluded in TPV or vehicle, whereas tdRFP-StaPL(ti)-NLS exhibits the inverse drug response. Bottom right, results were obtained in cells expressing reciprocal StaPL constructs. Representative images of live cells imaged 8-12 hrs after transfection. Scalebars, 15 um.

FIG. 3 shows that inserted between the modules of artificial zinc finger transciption factors, orthogonal StaPL modules permit bidirectional transcriptional modulation of the human VEGFA locus. (A) Top, schematics of drug-controllable synthetic transcription factors. StaPL modules separate a zinc finger (ZF) DNA binding domain (tagged with an HA epitope) from a FP marker and either a transcriptional activator (VPR) or repressor (KRAB), folowed by a NLS. Active NS3 will cleave off the ZF, and also the NLS. Bottom, constructs were transiently expressed in HEK293A for 48 hrs before immunoblotting. Bottom left, full-length ZF-StaPL(ai)-YFP-VPR (immunopositive for both HA and GFP) is only observed in the ASV condition, although it is weakly expressed. In DMSO and TPV, cleaved StaPL-YFP-VPR is observed. Bottom right, full length ZF-StaPL(ti)-tdRFP-KRAB (immunopositive for both HA and RFP) is only observed in TPV. In DMSO and ASV, cleaved StaPL-tdRFP-KRAB is observed. (B) ZF-StaPL effectors produce expected drug-dependent transcriptional regulation. ZF-StaPL(ai)-YFP-VPR and ZF-StaPL(ti)-tdRFP-KRAB were transiently expressed separately or coexpressed in HEK293A cells for 48 hrs and media supernatants were analyzed for secreted VEGF protein by ELISA. VEGF concentrations were averaged from 3 independent experiments and are expressed as the difference from empty vector control cells in respective drugs (average DMSO control, 716 pg/mL; ASV, 673 pg/mL; TPV, 930 pg/ml). Error bars, ±standard error of the mean.

FIG. 4 shows that dSpCas9 effectors enable bidirectional transcriptional regulation via internal StaPL domain insertion. (A) Sites in SpCas9 that permitted insertion of a StaPL sequence while maintaining dSpCas9 function in the presence of drug. (B) Schematics for a single-chain ASV-stabilized dSpCas9-based transcriptional repressor, a single-chain TPV-stabilized dSpCas9-based transcriptional activator, and the TRE3G-mCherry reporter cassette. (C) When KRAB-dSpCas9[StaPL(ai)1246] and VPR-dSpCas9[StaPL(ti)1246] were coexpressed along with sgRNA targeting the TRE3G locus, TPV alone resulted in activation of the reporter gene whereas ASV alone resulted in its repression, as expected. TPV and ASV together resulted in activation to a lesser degree than TPV alone. Cells stably expressing the reporter cassette were fixed 48 hrs after transfection with dSpCas9 effectors and sgRNA. GFP serves as marker for sgRNA expression. Scalebar, 200 um.

FIG. 5 shows that StaPL insertion between a tandem dimer of human Caspase-9 forms the basis of a cell suicide switch. (A) Schematic for Caspase-9 activation by chemical preservation of a StaPL(ai) linkage. An HA tag on the first caspase copy allows detection by immunoblot. (B) Proper cleavages were assessed by HA immunoblotting in cells incubated for 24 hrs in indicated drug. In HeLa Flp-In cells stably expressing catalytically inactive StaPL(ai)-dCasp9, the full-length tandem dimer is expressed in the presence of ASV, but not in the presence of DMSO or TPV. However, when cells expressing the active suicide switch are treated with ASV, no signal is detected by multiple antibodies, including antibodies to β-actin and to the coexpressed tRFP marker. This is consistent with widespread apoptosis in this condition. (C) Flow cytometry of live, annexin-stained stable StaPL(ai)-dCasp9 Flp-In HeLa cells reveals that a majority undergo apoptosis after 24 hrs incubation in ASV, as evidenced by high annexin stain signal. A second stable HeLa line expressing an inactive mutant serves as a control. Expression of tRFP under IRES is a marker for StaPL-dCasp9 expression. The parent Flp-In HeLa cell line assisted in confirming that the stable cells were tRFP positive. Circle delineates an area comprising 85% of the parent cell events and is reproduced for reference. Representative experiment is shown.

FIG. 6 shows development of orthogonal NS3 proteases. (A) Top, schematic of PSD95 fused to a SMASh cassette, which comprises a HCV NS3 protease cleavage site, a NS3 protease, and a degron. NS3 protease activity removes the degron, enabling PSD95 accumulation. Inhibition of the protease will prevent SMASh degron removal. Center and below, NS3 with a T54A mutation is more resistant to TPV inhibition than wild-type (wt) NS3 in transiently transfected HeLa cells (22 hrs expression), as evidenced by increased release of PSD95. ASV response is indistinguishable. Uncleaved (SMASh-tagged) protein has accelerated degradation but some as-yet-undegraded PSD95-SMASh is visible as an upper band. Center and bottom panels are derived from the same experiment/blot. (B) Effects of single NS3 mutations and combinations of multiple mutations on the ability of TPV and ASV to block SMASh degron removal were tested by transient transfections in HEK293A cells (22 hrs expression) and immunoblotting. Representative blots are shown. (C) Inhibition of protease by ASV and TPV was quantified from transfection and immunoblotting experiments as in (B), performed in triplicate. Error bars, ±standard error of the mean.

FIG. 7 shows that StaPL cleavages are dictated by the NS3 sequence and protease inhibitor present. (A) Generalized sequence of a StaPL cassette. Mutations present in the StaPL(ai) variant are in light grey (M, A, G), while mutations present in StaPL(ti) are in darker grey (L, K, R, Y). Left, cleavage sites (shown at N-terminal, but can be alternatively or additionally placed C-terminally to NS3 domain). TGCVVIVGRIVLSG, NS4A-derived cofactor strand. SGTS, artificial linker sequences. NSSPPAVTLTH, spacer sequence derived from the NS3 helicase domain. (B) StaPL(ai) and StaPL(ti) cleavages comport with microscopy results in FIG. 2. In HEK293A cells transiently transfected with tdYFP and/or tdRFP StaPL-NLS variants (8 hrs expression), cleavages are orthogonally controlled by ASV and TPV, and are regulated equally well on tdYFP versus tdRFP.

FIG. 8 shows optimization of the ZF-StaPL effectors. Top, initial architectures of the ZF-StaPL effectors. StaPL(ai) and StaPL(ti) sequences can be used to link a DNA-binding domain to transcriptional activation (p65) or inhibition (KRAB) domains. An HA tag on the ZF domain allows detection by immunoblot. Bottom, HEK293A cells transiently expressed indicated constructs for 12 hrs. Cleavage or preservation of the ZF domain proceeds as expected in each drug condition. ZF-StaPL effectors were assayed for activity by transfection into HEK293A and analysis of media supernatant for VEGF by ELISA. The repressor construct gave drug-dependent activity, whereas the activator did not, which necessitated replacing p65 with stronger activator VPR.

FIG. 9 shows that internal loops within target proteins can accommodate StaPL modules, making them drug regulable. Different sites within dSpCas9 were tested for their ability to tolerate an inserted StaPL(ti) module, such that it would permit protein function in the presence of TPV, but abolish dSpCas9 function in either ASV or DMSO (vehicle). Cells stably expressing the TRE3G-mCherry reporter cassette were fixed 48 hrs after transfection with dSpCas9 effectors±sgRNA targeting the TRE3G locus. GFP serves as marker for sgRNA expression. Scalebars, 200 um.

FIG. 10 shows performance of the StaPL-dCasp9 suicide switch. Bright-field images corroborate ASV induction of cell death in HeLa Flp-In cells stably expressing StaPL(ai)-Casp9. After 24 hrs incubation, visible rounding and lifting occurs with ASV. Cells expressing catalytically inactive StaPL-dCasp9 serve as control. Scalebar, 500 um.

FIG. 11 Kinetics, reversibility, and dose responsiveness of StaPL-mediated transcriptional activators. (a) Activation time courses are similar between VPR-dCas9(StaPLTI) and constitutive VPR-dCas9 after transient transfection in HEK293-TRE3G-mCherry cells. (b) Kinetics of mCherry RFP transcriptional activation by VPR-dCas9(StaPLTI) in 10 μM TPV or in DMSO vehicle control, as measured by RT-qPCR. (c) Kinetics of VEGFA transcriptional activation by ZFVEGFA-StaPLAI-YFP-VPR in 1 μM ASV or in DMSO vehicle control. (d,e) RFP transcriptional activation induced by VPR-dCas9(StaPLTI), sgRNA, and TPV in HEK293-TRE3G-mCherry cells (d) is not fully reversible after drug washout, while VEGFA transcriptional activation induced by ZFVEGFA-StaPLAI-YFP-VPR and ASV in HEK293A cells (e) is reversible, reflecting the different mechanisms of action of TPV and ASV. (f) Dose-response relationship between TPV and mCherry RFP transcriptional activation by VPR-dCas9(StaPLTI) and sgRNA. HEK293-TRE3G-mCherry cells expressed VPR-dCas9(StaPLTI) and sgRNA for 48 h in indicated drug condition. (g) Dose-response relationship between ASV and VEGFA transcriptional activation by ZFVEGFA-StaPLAI-YFP-VPR.

### DEFINITIONS

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in, corresponding portions of the produced RNAs (e.g., hnRNAs, mRNAs, rRNAs, tRNAs).

Where the phrase “amino acid sequence” is recited herein to refer to an amino acid sequence of a peptide or protein molecule, amino acid sequence and like terms, such as polypeptide or protein are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “sample” is used in its broadest sense. In one sense, it is meant to include cells (e.g., human, bacterial, yeast, and fungi), an organism, a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, crystals and industrial samples. Such examples are not however to be construed as limiting the sample types applicable to the present disclosure.

As used herein, the term “organism” refers to any entity from which total genomic DNA and/or RNA can be derived. For example, organisms may be subjects, strains, isolates, or species. In some embodiments, a subject, strain, isolate or species may be selected from humans, bacteria, viruses, yeast, algae, fungi, animals and plants.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the term “container” is used in its broadest sense, and includes any material useful for holding a sample, reagent, or organism. A container need not be completely enclosed. Containers include tubes (e.g., eppendorf or conical tubes), plates, wells, microtiter plate wells, or any material capable of separating one sample from another (e.g., a microfluidic channel or engraved space on a solid surface). Such examples are not however to be construed as limiting the containers applicable to the present disclosure.

As used herein, the term “kit” refers to any delivery system for delivering materials. In the context of reaction assays, such delivery systems include systems that allow for the storage, transport, or delivery of reagents (e.g., cloning vectors, protein controls, enzymes, etc. in the appropriate containers) and/or supporting materials (e.g., buffers, written instructions for performing cloning and expression etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain a microarray for use in an assay, while a second container contains oligonucleotides. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “fusion protein” refers to a polypeptide comprising multiple (e.g., two or more) distinct polypeptides, regardless of their relative location within the polypeptide.

### DETAILED DESCRIPTION

Provided herein are compositions and methods for inducing protein function. For example, in some embodiments, provided herein are compositions and methods for pharmacological induction of protein function.

Experiments described herein demonstrate single-chain drug-inducible proteins by insertion of stabilizable polypeptide linkages (StaPLs). In some embodiments, a StaPL sequence is a combination of a sequence-specific protease domain and a cognate substrate site that is inserted within a polypeptide to cause its separation into non-functional fragments by default. In some embodiments, the StaPLs described herein use the hepatitis C virus (HCV) NS3 protease, for which multiple clinically available inhibitors exist'. When protein function is desired, application of an inhibitor blocks proteolysis of the polypeptide, allowing full-length protein to undergo maturation. Experiments show StaPL sequences can be used to confer functional inducibility onto synthetic modular transcriptional regulators, CRISPR/Cas9-based transcriptional regulators, and Caspase-9, establishing the generalizability of this approach. Further experiments show the ability of two orthogonal StaPL sequences to control transcriptional activators and repressors in the same cell, demonstrating the use of multiplexed pharmacological induction of protein activity using StaPLs.

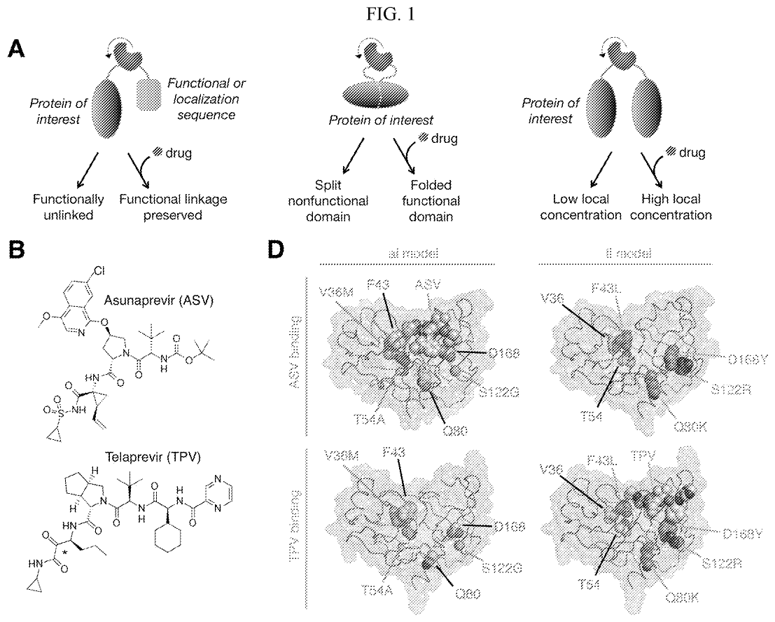

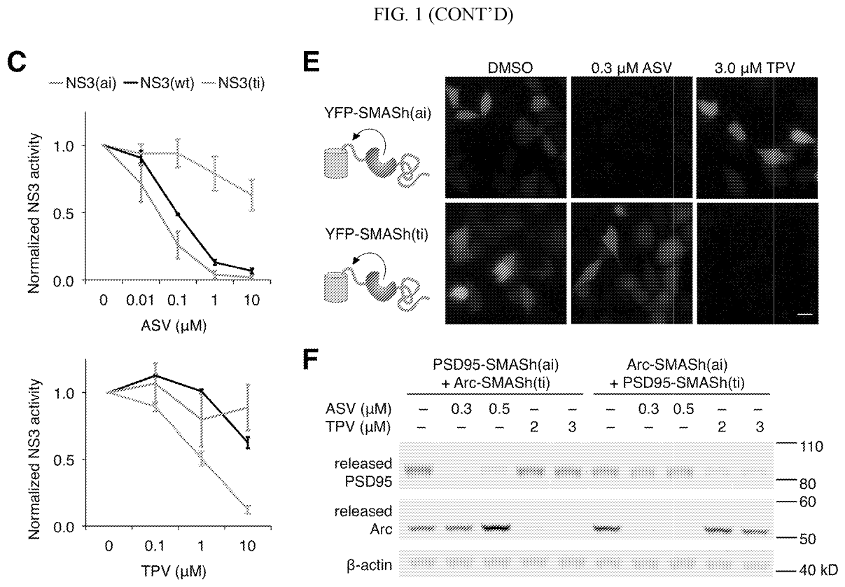

Accordingly, in some embodiments, provided herein is a fusion protein comprising a) a first polypeptide of interest; and b) a first protease and a substrate for the protease. In some embodiments, the protease and the substrate are inserted between multiple domains of the polypeptide. In some embodiments, the protease and the substrate are inserted within a domain of the polypeptide of interest. In some embodiments, the protease and the substrate are inserted between multiple (e.g., two) copies of the polypeptide of interest (e.g., a protein that dimerizes or forms multimers in its functional state). The present disclosure is not limited to the configurations described herein (See e.g., FIG. 1A). Any conformation that functions to form active proteins or polypeptides or peptide when proteolysis activity of the protease is inhibited is specifically contemplated.

In some embodiments, multiple fusion proteins are utilized in order to provide multiplex protein activation. In some embodiments, a second (or more) fusion protein is provided. In some embodiments, the second fusion protein comprises a second polypeptide of interest and a second protease, wherein the second protease is distinct from said first protease. In some embodiments, the first protease and the second protease are inhibited by different protease inhibitors.

The present disclosure is not limited to particular proteases. In some embodiments, the first protease is an HCV NS3 protease (see e.g., NCBI Reference Sequence: NP_803144.1; SEQ ID NO:1 (amino acids 1-193 of NP_803144.1); apitayaqqt rgllgciits ltgrdknqve gevqivstat qtflatcing vcwtvyhgag trtiaspkgp viqmytnydq dlvgwpapqg srsltpctcg ssdlylvtrh advipvrrrg dsrgsllspr pisylkgssg gpllcpagha vglfraavct rgvakavdfi pvenlettmr spvftdnssp pay). In some embodiments, the protease comprises at least one mutation. For example, in some embodiments, the first HCV NS3 protease comprises V36M, T54A, and S122G mutations and the second HCV NS3 protease comprises F43L, Q80K, S122R, and D168Y mutations, although other mutations are specifically contemplated. In some embodiments, the HCV NS3 protease comprising V36M, T54A, and S122G mutations is resistant to telaprevir (TPV) and sensitive to asunaprevir (ASV) and the HCV NS3 protease comprising F43L, Q80K, S122R, and D168Y mutations is resistant to ASV and sensitive to TPV.

In some embodiments, mutations that reduce immunogenicity of the proteases are employed (e.g., for gene therapeutic usage in human patients), while maintaining sufficient protease activity. Such mutations for the HCV NS3 protease include one or more of G15R, 118V, S20N, V55A, Y105A, L106A, H110A, A111G, V113A, V151A, 1170V, and V172A (see e.g., Soderholm and Sallberg, J. Infect. Dis., 194(12), 1724-8 (2006); and Soderholm et al., Gut, 55(2), 266-74, (2006), each of which is herein incorporated by reference in its entirety).

In some embodiments, the protease is a West Nile Virus NS3 protease (also known as NS2B/NS3 protease) with associated inhibitors (see e.g., Behnam, et al., J. Med. Chem. 58, 9354-9370 (2015); herein incorporated by reference in its entirety). In some embodiments, the protease is a Zika Virus NS3 protease (also known as NS2B/NS3 protease) with associated inhibitors (see e.g., Li et al., Cell Res. (2017) July 7, herein incorporated by reference in its entirety). In some embodiments, the protease is a Dengue Virus NS3 protease (also known as NS2B/NS3 protease) with associated inhibitors (see e.g., Boldescu, et al., Nat Rev Drug Discov (2017), herein incorporated by reference in its entirety). In some embodiments, the protease is a Severe Acute Respiratory Syndrome (SARS) virus 3CLpro protease, with associated inhibitors (e.g., Pillaiyar, J. Med. Chem. 59, 6595-6628 (2016), herein incorporated by reference in its entirety). In some embodiments, the protease is a Human Rhinovirus (HRV) 3C protease, with associated inhibitors (e.g., Witherell, Curr Opin Investig Drugs 1, 297-302 (2000), herein incorporated by reference in its entirety).

Moreover, as described above, variant forms of proteases find use in the compositions and methods described herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present disclosure provide variants of proteases disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer ed., Biochemistry, pg. 17-21, 2nd ed, WH Freeman and Co., 1981). Whether a change in the amino acid sequence of a peptide results in a functional polypeptide can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

Variants may be produced by methods such as directed evolution or other techniques for producing combinatorial libraries of variants. In still other embodiments, the nucleotide sequences encoding a protease may be engineered in order to alter a coding sequence including, but not limited to, alterations that modify the cloning, processing, localization, secretion, and/or expression of the gene product. For example, mutations may be introduced using available techniques (e.g., site-directed mutagenesis to insert new restriction sites, alter glycosylation patterns, or change codon preference, etc.).

The present disclosure is not limited to particular polypeptides of interest. In some embodiments, the polypeptide of interest is, for example, a transcription factor, a hormone, an enzyme, a regulatory protein, a nuclease enzyme, a protease enzyme, or a metabolic enzyme.

Further embodiments provide a nucleic acid encoding the fusion proteins described herein. In some embodiments, the nucleic acid is on a vector.

In some embodiments, the present disclosure provides vectors and recombinant expression systems for expressing fusion proteins described herein (e.g., in a cell). The present disclosure is not limited to particular expression vectors. Exemplary vectors and expression methods are described herein.

In some embodiments, proteins are expressed using any suitable vector and expression system. In some embodiments, peptides are expressed in bacterial or eukaryotic expression vectors (e.g., commercially available vectors). In some embodiments, peptides are expressed in retroviral (e.g., adeno, adeno-associated, or lenti-viral vectors). Suitable vectors are introduced into suitable host cells (e.g., bacterial or eukaryotic host cells), expression is induced, and peptides are isolated using any suitable method.

The production of a recombinant retroviral vector carrying a gene of interest is typically achieved in two stages. First, the gene of interest is inserted into a retroviral vector which contains the sequences necessary for the efficient expression of the gene of interest (including promoter and/or enhancer elements which may be provided by the viral long terminal repeats [LTRs] or by an internal promoter/enhancer and relevant splicing signals), sequences required for the efficient packaging of the viral RNA into infectious virions (e.g., the packaging signal [Psi], the tRNA primer binding site [−PBS], the 3′ regulatory sequences required for reverse transcription [+PBS] and the viral LTRs). The LTRs contain sequences required for the association of viral genomic RNA, reverse transcriptase and integrase functions, and sequences involved in directing the expression of the genomic RNA to be packaged in viral particles. For safety reasons, many recombinant retroviral vectors lack functional copies of the genes that are essential for viral replication (these essential genes are either deleted or disabled); the resulting virus is said to be replication defective or incompetent.

Second, following the construction of the recombinant vector, the vector DNA is introduced into a packaging cell line. Packaging cell lines provide viral proteins required in trans for the packaging of the viral genomic RNA into viral particles having the desired host range (i.e., the viral-encoded gag, pol and env proteins). The host range is controlled, in part, by the type of envelope gene product expressed on the surface of the viral particle. Packaging cell lines may express ecotrophic, amphotropic or xenotropic envelope gene products. Alternatively, the packaging cell line may lack sequences encoding a viral envelope (env) protein. In this case the packaging cell line will package the viral genome into particles that lack a membrane-associated protein (e.g., an env protein). In order to produce viral particles containing a membrane associated protein that will permit entry of the virus into a cell, the packaging cell line containing the retroviral sequences is transfected with sequences encoding a membrane-associated protein (e.g., the G protein of vesicular stomatitis virus [VSV]). The transfected packaging cell will then produce viral particles that contain the membrane-associated protein expressed by the transfected packaging cell line; these viral particles, which contain viral genomic RNA derived from one virus encapsidated by the envelope proteins of another virus are said to be pseudotyped virus particles.

Commonly used recombinant retroviral vectors are derived from the amphotropic Moloney murine leukemia virus (MoMLV) (Miller and Baltimore, Mol. Cell. Biol., 6:2895 [1986]). The MoMLV system has several advantages: 1) this specific retrovirus can infect many different cell types, 2) established packaging cell lines are available for the production of recombinant MoMLV viral particles and 3) the transferred genes are permanently integrated into the target cell chromosome. The established MoMLV vector systems comprise a DNA vector containing a small portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle assembly (Markowitz et al., J. Virol., 62:1120 [1998]).

Other commonly used retrovectors are derived from lentiviruses including, but not limited to, human immunodeficiency virus (HIV) or feline immunodeficiency virus (FIV). Lentivirus vectors have the advantage of being able to infect non replicating cells.

The low titer and inefficient infection of certain cell types by retro vectors has been overcome by the use of pseudotyped retroviral vectors which contain the G protein of VSV as the membrane associated protein. Unlike retroviral envelope proteins which bind to a specific cell surface protein receptor to gain entry into a cell, the VSV G protein interacts with a phospholipid component of the plasma membrane (Mastromarino et al., J. Gen. Virol., 68:2359 [1977]). Because entry of VSV into a cell is not dependent upon the presence of specific protein receptors, VSV has an extremely broad host range. Pseudotyped retroviral vectors bearing the VSV G protein have an altered host range characteristic of VSV (i.e., they can infect almost all species of vertebrate, invertebrate and insect cells). Importantly, VSV G-pseudotyped retroviral vectors can be concentrated 2000-fold or more by ultracentrifugation without significant loss of infectivity (Burns et al., Proc. Natl. Acad. Sci. USA, 90:8033 [1993]).

The VSV G protein has also been used to pseudotype retroviral vectors based upon the human immunodeficiency virus (HIV) (Naldini et al., Science 272:263 [1996]). Thus, the VSV G protein may be used to generate a variety of pseudotyped retroviral vectors and is not limited to vectors based on MoMLV.

The majority of retroviruses can transfer or integrate a double-stranded linear form of the virus (the provirus) into the genome of the recipient cell only if the recipient cell is cycling (i.e., dividing) at the time of infection. Retroviruses that have been shown to infect dividing cells exclusively, or more efficiently, include MLV, spleen necrosis virus, Rous sarcoma virus human immunodeficiency virus, and other lentiviral vectors.

In some embodiments, the nucleic acid is expressed in an expression cassette. In particular embodiments, the expression cassette is a eukaryotic expression cassette. The term “eukaryotic expression cassette” refers to an expression cassette which allows for expression of the open reading frame in a eukaryotic cell. A eukaryotic expression cassette comprises regulatory sequences that are able to control the expression of an open reading frame in a eukaryotic cell, preferably a promoter and polyadenylation signal. Promoters and polyadenylation signals included in the recombinant DNA molecules are selected to be functional within the cells of the subject to be immunized. Examples of suitable promoters include but are not limited to promoters from cytomegalovirus (CMV), such as the strong CMV immediate early promoter, Simian virus 40 (SV40), Mouse Mammary Tumor Virus (MMTV), Human Immunodeficiency Virus (HIV), such as the HIF Long Terminal Repeat (LTR) promoter, Moloney virus, Epstein Barr Virus (EBV), and from Rous Sarcoma Virus (RSV) as well as promoters from human genes such as human actin, human myosin, human hemoglobin, human muscle creatine, and human metallothionein.

Examples of suitable polyadenylation signals include but are not limited to the bovine growth hormone (BGH) polyadenylation site, SV40 polyadenylation signals and LTR polyadenylation signals.

Other elements can also be included in the recombinant DNA molecule. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the cell or organism utilized. The person skilled in the art can produce recombinant DNA molecules that are functional in a given subject species.

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

In some embodiments, CRISPR/Cas9 systems are used to incorporate fusion proteins into cells. Clustered regularly interspaced short palindromic repeats (CRISPR) are segments of prokaryotic DNA containing short, repetitive base sequences. These play a key role in a bacterial defence system, and form the basis of a genome editing technology known as CRISPR/Cas9 that allows permanent modification of genes within organisms.

By delivering the Cas9 nuclease complexed with a synthetic guide RNA (gRNA) into a cell, the cell's genome can be cut at a desired location, allowing existing genes to be removed and/or new ones added.

Still other embodiments provide a kit or system, comprising: a) a nucleic acid as described herein; and b) at least one protease inhibitor. In some embodiments, kits provide components useful, necessary, or sufficient for generating and using cells that express the fusion proteins described herein. In some embodiments, kits comprise additional components (e.g., controls, buffers, vectors, detection reagents for detecting protein function and/or expression, etc.). In some embodiments, components of a kit are provided in one or more containers that each comprise a single or multiple components.

Certain embodiments provide a method of modulating the activity or function of a polypeptide of interest, comprising: contacting the cell as described herein with a protease inhibitor under conditions such that the polypeptide of interest is active.

The compositions, kits, systems, and method described herein find use in a variety of research, screening, and clinical applications. In some embodiments, the fusion proteins are used to study protein function and activity (e.g., in vitro or in a non-human animal). In some embodiments, the fusion proteins are used to screen test compounds (e.g., protease inhibitors) for activity. In some embodiments, the fusion proteins are used in vivo to modulate function of a protein of interest (e.g., to treat a disease or condition). In some embodiments, the composition described herein find use in CAR-T gene therapy and/or allogeneic hematopoietic stem cell transplantation, for example, to provide a Caspase-9 suicide switch gene in order to ameliorate possible negative outcomes (e.g., cytokine storm or graft-versus-host-disease, respectively).

### EXPERIMENTAL

### Example 1

Previous assays used HCV NS3 protease and its small-molecule inhibitors to control new protein visualization and production with drug. In the TimeSTAMP method, proteins of interest are encoded fused to a tag and a cis-cleaving HCV NS3 protease domain to remove that tag by default, but addition of NS3 protease inhibitor enables newly synthesized protein copies to be identified by the tag (Butko, M. T. et al. Nat Neurosci 15, 1742-1751 (2012); Lin, M. et al., Proc. Natl. Acad. Sci. U.S.A. 105, 7744-7749 (2008). SMASh is a degron whose removal by a cis-cleaving HCV NS3 protease domain can be inhibited by a drug, enabling HCV protease inhibitors to shut off protein production (Chung, H. K. et al. Nat Chem Biol 11, 713-720 (2015)).

This example describes a method of preserving polypeptide linkage by pharmacological inhibition of a cis-cleaving protease that can be used as a general method to induce protein function in at least three different ways (FIG. 1A). First, an autoproteolytic tag is placed between a protein of interest and a functional domain, so the two functions are unlinked by default but are linked when proteins are synthesized in the presence of protease inhibitor (left). Second, the autoproteolytic tag is placed in a linker within a domain, so the domain is split into two fragments by default but matures correctly if synthesized in the presence of protease inhibitor (middle). Third, the autoproteolytic tag is placed between two copies of a protein so that it is expressed as a monomer normally, but is effectively a tandem dimer when synthesized in the presence of inhibitor (right). For proteins that are activated by dimerization, preservation of linkage in the presence of protease inhibitor would then lead to protein activation. These drug-preservable connections between protein domains or protein fragments are referred to as stabilizable polypeptide linkages (StaPLs).

As gene- and cell-based therapies become more complex, the ability to control multiple protein activities in a single cell, or control a single pathway in opposite directions, is highly desirable. For example, it is sometimes useful to activate a pathway beyond its endogenous level at some times and repress it at other times. The StaPL-system finds use to control multiple proteins or multiple outputs from a single protein. To control two outcomes in the same cell independently, two sequence-specific proteases that are inhibited by two different drugs are used. To achieve this, for example, two variants of the HCV NS3 protease domain (hereafter referred to as simply NS3) that are inhibited by different drugs were developed, in effect diverging NS3 into two operational species defined by drug sensitivity.

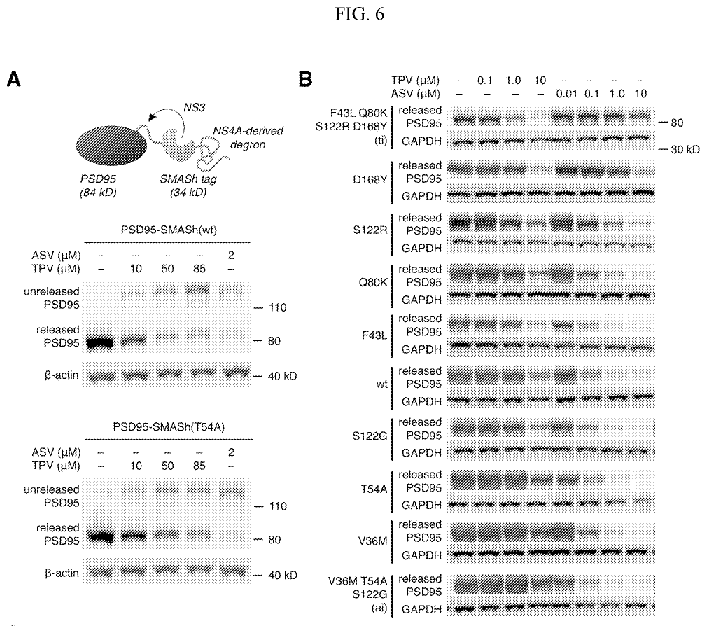

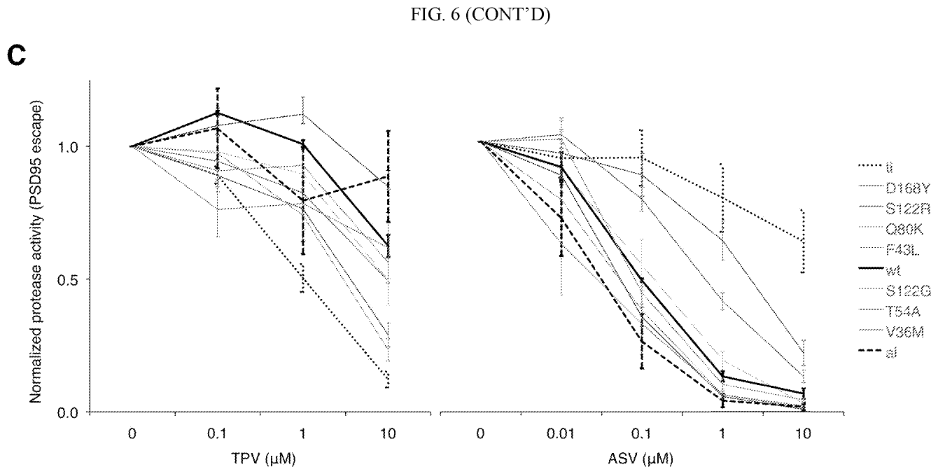

To develop an orthogonal set of NS3-drug interactions, mutants of HCV NS3 protease known to affect inhibition of HCV replication by telaprevir (TPV) or asunaprevir (ASV, Table 1, FIG. 1B) were used. One mutation previously found to cause resistance to TPV but not ASV in the HCV replicon assay, T54A (McPhee, F. et al. Antimicrob. Agents Chemother. 56, 5387-5396 (2012)), was investigated. It was found that a PSD95-SMASh construct with the T54A mutation in the NS3 sequence of a SMASh tag showed higher levels of released PSD95 in the presence of TPV than a PSD95-SMASh construct with position 54 reverted to Thr (FIG. 6A). This indicated that NS3 T54A retained more protease activity than wild-type NS3 in the presence of TPV, consistent with the observation of NS3 T54A resistance to TPV in a replicon assay. In addition, SMASh control by ASV was equally effective with wild-type NS3 or NS3 T54A, also consistent with the behavior of T54A mutants in the replicon assay (FIG. 6A). These results describe a simple assay for NS3 sensitivity to inhibitors based on SMASh, where sensitivity is revealed by inhibition of SMASh tag removal and induction of protein degradation. Using this assay, three mutations were found to cause NS3 resistance to TPV without affecting inhibition by ASV, and four mutations found to cause resistance to ASV without affecting inhibition by TPV (McPhee, F. et al. Antimicrob. Agents Chemother. 56, 3670-3681 (2012)). A triple mutant (V36M T54A S122G) was highly resistant to TPV and more sensitive to ASV compared to wild-type NS3, while a quadruple mutant (F43L Q80K S122R D168Y) was highly resistant to ASV and more sensitive to TPV (FIG. 6B,C, FIG. 6C). These two mutants were termed NS3(ai) and NS3(ti), respectively, for ASV-inhibited and TPV-inhibited, respectively.

The atomic structure of the NS3 protease domain was examined to understand the chemical basis for NS3(ai) and NS3(ti) orthogonality. The four mutations in NS3(ti) are all located near the inhibitor-binding pocket and involve substitutions to larger or more globular side chains, indicating that they blocked ASV binding by shrinking the binding pocket (FIG. 1D). This same shrinking of the binding pocket may have improved TPV binding, as the more linear TPV does not fill the binding pocket of wild-type NS3 as completely as ASV (Romano, K. P. et al., PLoS Pathog 8, e1002832 (2012); Soumana, D. I., Ali, A. & Schiffer, C. A. ACS Chem Biol 9, 2485-2490 (2014). In contrast, the NS3 structure shows that the TPV resistance of NS3(ai) is likely to result from a reduced ability to form a covalent complex with TPV, an important component of the mechanism of action of TPV (Romano et al., supra). The T54A mutation in NS3ai is known to reduce the enzymatic catalytic rate (Tong, X. et al. Antiviral Res 70, 28-38 (2006)), and thus is expected to hinder formation of the covalent TPV complex as well. Although a V36M mutation alone does not affect NS3 catalysis rates (Zhou, Y. et al., Antimicrob. Agents Chemother. 52, 110-120 (2008)), the side chains at positions 63 and 54 directly face each other (FIG. 1D), so it is possible that V36M in the context of T54A causes a further impairment of catalytic activity. The improved inhibition of NS3(ai) by ASV may then be explained by reduced ability of NS3(ai) to cleave substrates in the short intervals between ASV dissociation and reassociation.

To further confirm functional orthogonality of NS3(ai)-ASV and NS3(ti)-TPV in cells, SMASh tags based on NS3(ai) or NS3(ti) were used to regulate YFP production in cells. YFP-SMASh(ai) expression was suppressed only by ASV while YFP-SMASh(ti) expression was suppressed only by TPV (FIG. 1E). It was also shown that SMASH(ai) and SMASH(ti) enabled orthogonal regulation of the production of two different proteins in the same cell orthogonally (FIG. 1F). Here, SMASh(ai) responded well to 0.3 μM ASV, while SMASh(ti) responded well to 3 μM TPV.

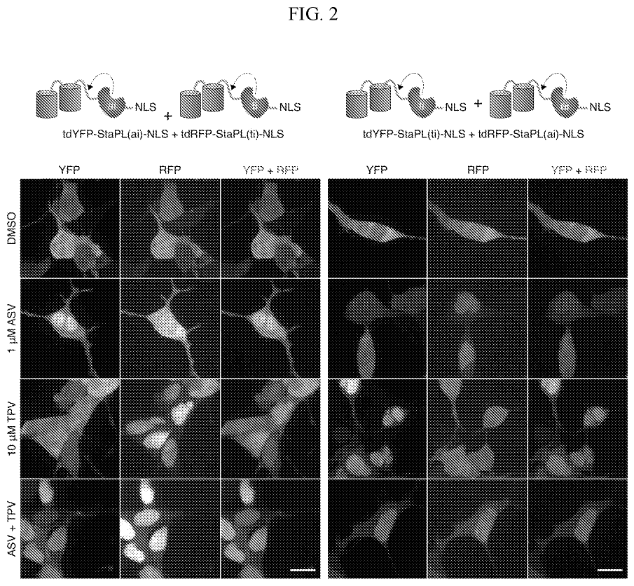

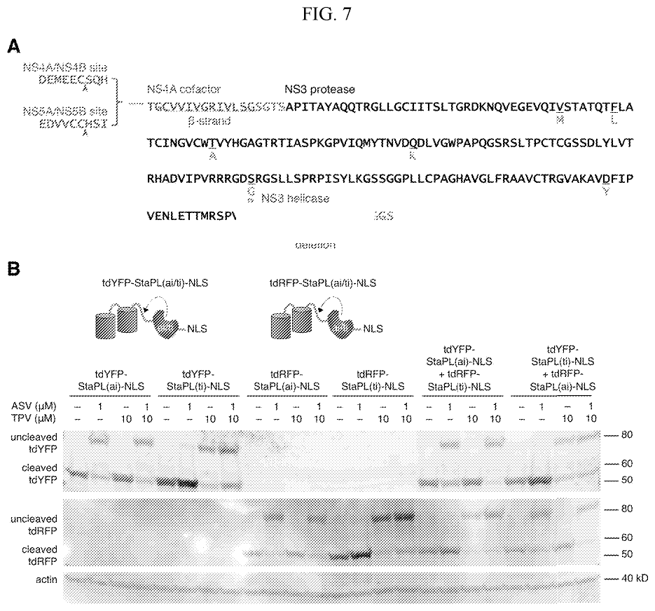

Regulation of the linkage of functional output domains in a synthetic chimeric protein, as postulated above (FIG. 1A) was investigated. For a stabilizable polypeptide linkage (StaPL) sequence, a HCV NS3 protease cleavage site derived from the NS4A/4B junction, followed by the NS4A-derived cofactor strand (to enhance NS3 protease activity) and either NS3(ai) or NS3(ti) were used (FIG. 7A). The StaPL(ai) and StaPL(ti) sequences undergo self-removal in the absence of drug, but removal is inhibited by ASV and TPV, respectively. In the presence of drug, the StaPL sequence is retained and serves to link the two domains together (FIG. 2). A functional tag and a nuclear localization sequence (NLS) were attached to two different proteins, a tandem dimeric Venus YFP (abbreviated tdYFP) and a tandem dimeric Tomato RFP (tdRFP), via StaPL(ai) and StaPL(ti) sequences, respectively. Production of each full-length protein occurred specifically in the presence of the appropriate drug (FIG. 7B). Nuclear localization of tdVenus and tdTomato were induced orthogonally by ASV and TPV (FIG. 2).

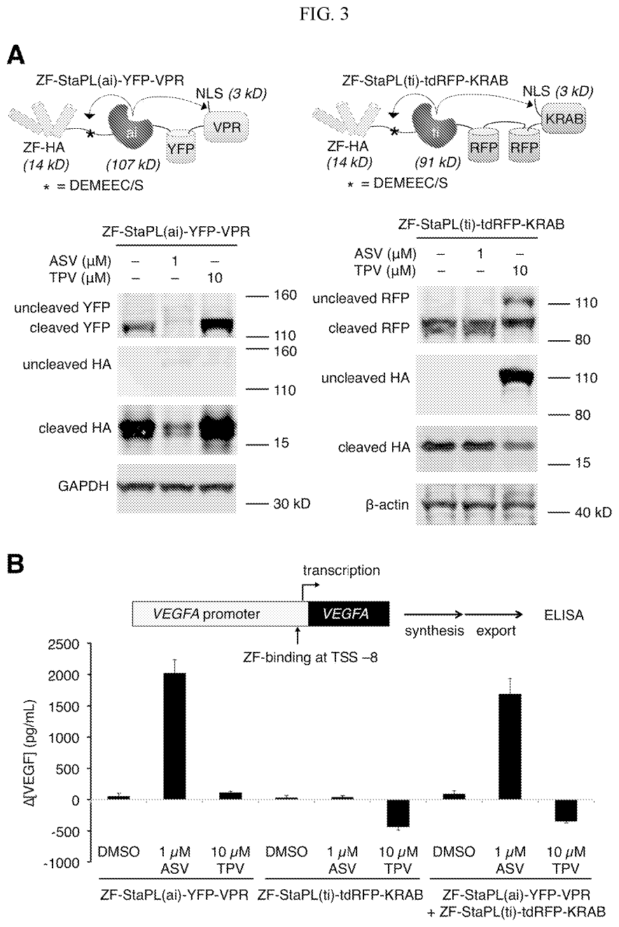

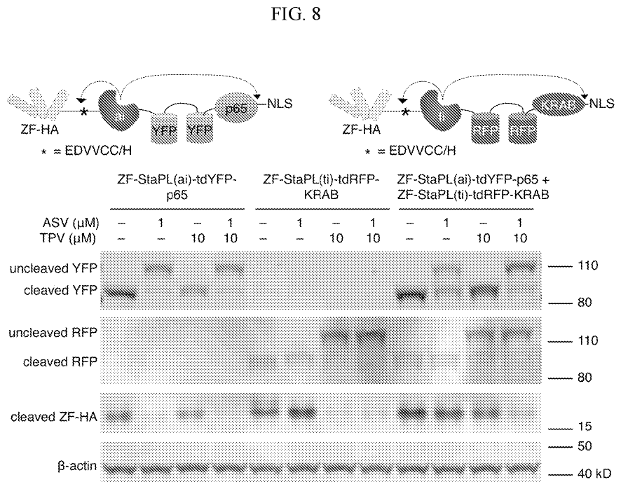

It was next determined whether StaPL sequences functioned to bidirectionally control gene transcription. Vascular endothelial growth factor (VEGF) was regulated as VEGF production is therapeutically useful for treatment of conditions involving vascular insufficiency (Giacca, M. & Zacchigna, S. Gene Ther 19, 622-629 (2012)), but uncontrolled production can exacerbate diseases thaty on angiogenesis such as wet macular degeneration and cancer (Ferrara, N. & Adamis, A. P. Nat Rev Drug Discov 15, 385-403 (2016)). Combinations of a zinc-finger (ZF) DNA-binding domain targeting the VEGF-A locus (Liu, P. Q. et al. J. Biol. Chem. 276, 11323-11334 (2001)), a StaPL(ti) or StaPL(ai) cassette, and a regulatory effector were constructed. The regulatory domains tested were the tdRFP fused to the transcriptional repressor domain KRAB (Lupo, A. et al. Curr Genomics 14, 268-278 (2013)), tdYFP fused to the transcriptional activation domain of p65, or YFP fused to the VP64-p65-Rta (VPR) transcriptional activation sequence (Chavez, A. et al. Nat Methods 12, 326-328 (2015)). When expressed in cells, each construct showed separation of ZF and regulatory domains in the absence of its specific inhibitor, and preservation of the full-length fusion only in the presence of its specific inhibitor (FIG. 8, FIG. 3A). ZF-StaPL(ti)-tdRFP-KRAB and ZF-StaPL(ai)-YFP-VPR were chosen for further testing on their ability to regulate VEGF production from the endogenous gene. After co-transfection of HEK293A cells with these constructs, VEGF amounts in the culture medium were increased in ASV and repressed in TPV (FIG. 3B), demonstrating that two orthogonal StaPL sequences enable bidirectional control of transcriptional outputs from a common DNA-binding domain.

In the examples above, the StaPL sequence serves as a drug-stabilized link between domains in a synthetic chimeric protein. The StaPL method was next extended to natural loops within domains to regulate proteins that are otherwise native in structure. Enzymatically deficient Cas9 (dCas9) was used to confirm this method. Several methods for drug regulation of Cas9 or dCas9 function have been described. Fusion of the destabilizing ddFKBP domain to Cas9 enables the chemical Shield to increase Cas9 levels, but inducibility is limited by substantial expression in the absence of Shield (Geisinger, J. M., et al., Nucleic Acids Res. 44, e76 (2016)). In one two-component method, dCas9 is split into two fragments which are then fused to domains that associate upon drug addition, so that DNA binding and transcriptional regulation are induced by drug, but this system is also limited in dynamic range (Zetsche et al., supra). More effective two-component systems were recently developed in which dCas9 and transcriptional regulatory domains are fused to domains that associate upon drug addition, so that dCas9 is always bound to DNA but transcriptional activation or further repression is induced by drug (Gao, Y. et al. Complex transcriptional modulation with orthogonal and inducible dCas9 regulators. Nat Methods 13, 1043-1049 (2016)). However, controlling both activation and repression in the same cells using this approach requires two different chemical dimerizers and the expression of four polypeptides (Gao et al., supra), which is cumbersome.

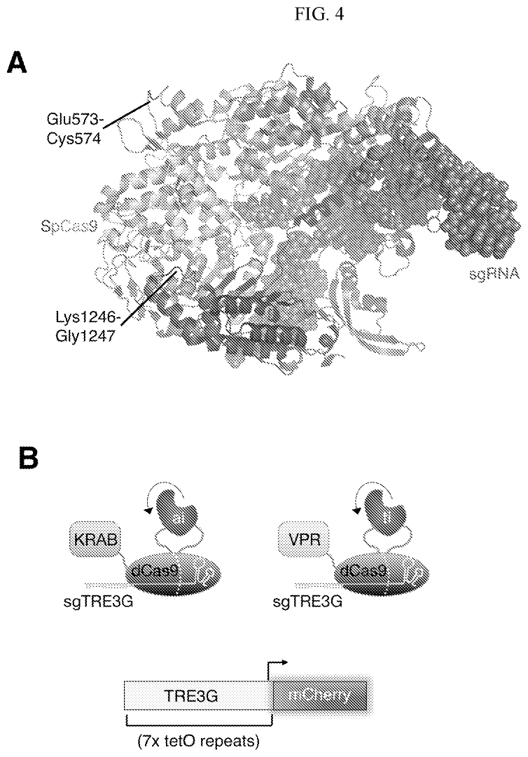

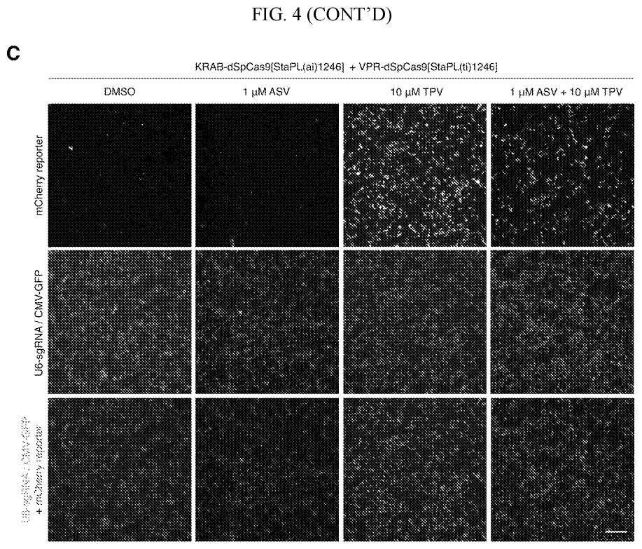

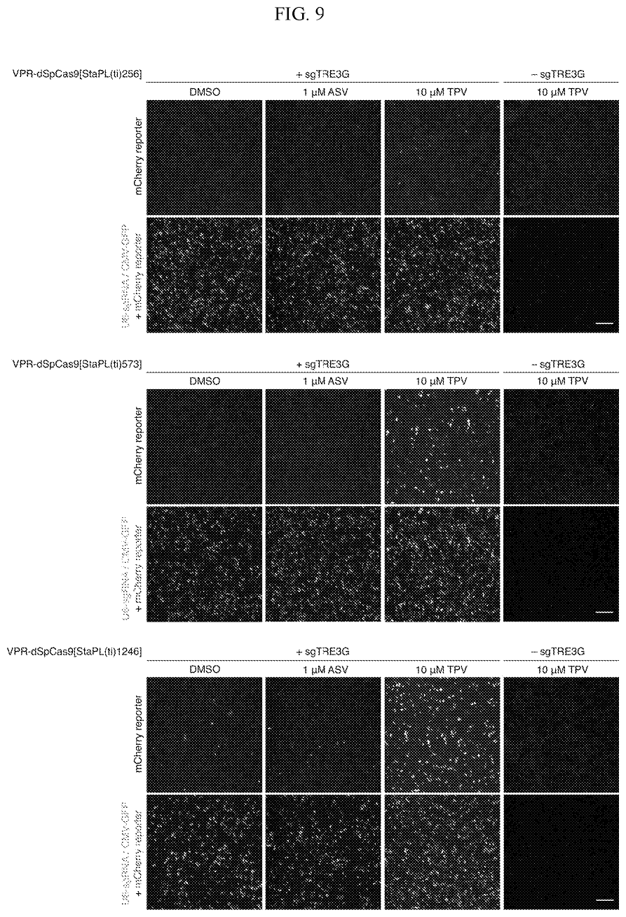

It was explored whether single-chain drug-inducible dCas9 variants could be constructed using StaPL sequences. To make drug-inducible variants of enzymatically deficient S. pyogenes Cas9 (dSpCas9), a StaPL(ti) sequence was inserted into three non-conserved loop positions within a dSpCas9 protein bearing the VPR transactivation sequence at its N-terminus (VPR-dSpCas9). To assay for transcriptional activation in the absence or presence of TPV, constructs were expressed in cells stably transfected with an mCherry reporter driven by a promoter containing tetracycline repressor elements (TREs), also coexpressing a guide RNA targeting the TRE elements. It was contemplated that, in the absence of TPV, the self-removal of the protease would cleave the dSpCas9 domain into two, preventing maturation of the protein and thereby DNA binding. In contrast, in the presence of TPV, cleavage would not happen and the StaPL sequence would be retained as an internal fusion at the loop positions. Two out of three insertion sites screened, after amino acids 573 and 1246 in loops within the REC2 and PI domains of dSpCas9 (FIG. 4A), supported robust drug-dependent transcriptional activation by TPV (FIG. 9). A VPR-dSpCas9 protein with the StaPL(ti) sequence inserted at aa 1246 is referred to as VPR-dSpCas9[StaPL(ti)1246]. By replacing VPR with a KRAB transcriptional repressor domain and StaPL(ti) with StaPL(ai), KRAB-dSpCas9[StaPL(ai)1246], which functions as a transcriptional repressor in the presence of ASV was constructed. VPR-dSpCas9[StaPL(ti)1246] nd KRAB-dSpCas9[StaPL(ai)1246] thus allow bidirectional regulation of transcription by TPV and ASV (FIG. 4B). Indeed, the activating and repressing functions of VPR-dSpCas9[StaPL(ti)1246] and KRAB-dSpCas9[StaPL(ai)1246] were induced in cells by TPV and ASV, respectively, in an orthogonal manner (FIG. 4C).

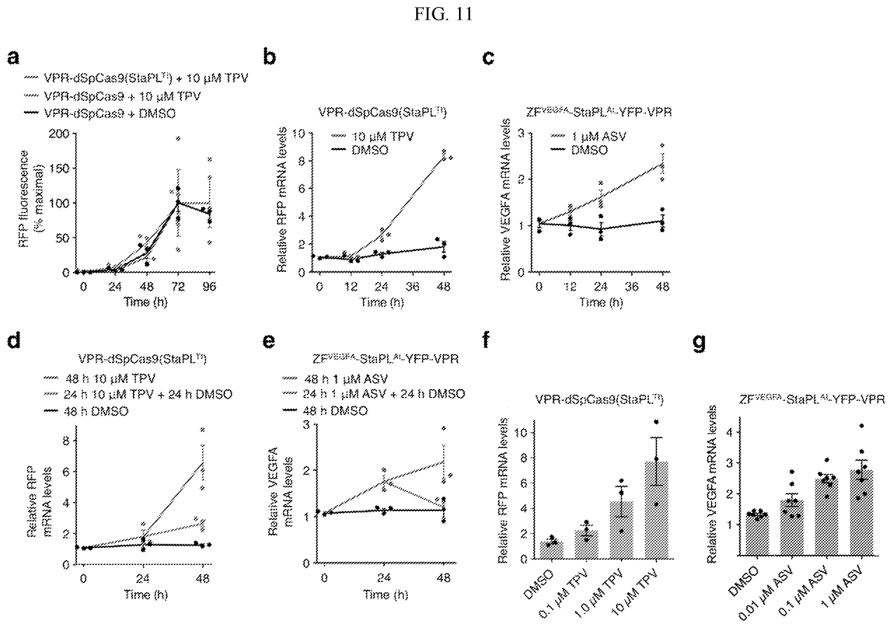

Next, the kinetics, reversibility, and dose responsiveness of transcriptional activation by StaPL was investigated. Kinetics were consistent with the accumulation of new intact protein copies in the presence of drug (FIG. 11 9a-c). Reversal was not observed 24 h after the removal of TPV from VPR-dSpCas9(StaPLTI), but was observed after the removal of ASV from ZFVEGFA-StaPLAI-YFP-VPR, consistent with the known covalent mechanism of action of TPV and noncovalent mechanism of action of ASV (FIG. 11d,e). The dose responsiveness of VPR-dSpCas9(StaPLTI) and ZFVEGFA-StaPLAIYFP-VPR mirrored that of SMAShTI and SMAShTI, respectively (FIG. 11f,g and Tables 2 and 3), indicating consistent drug responsiveness of the NS3TI and NS3AI proteases in different contexts.

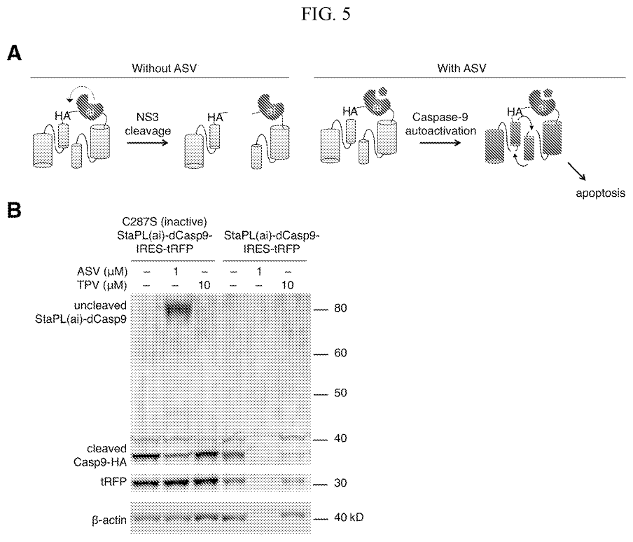

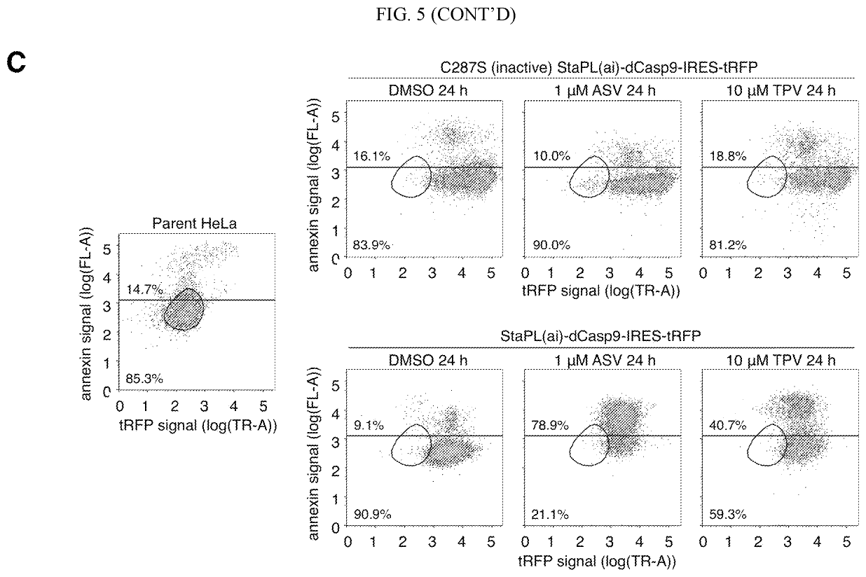

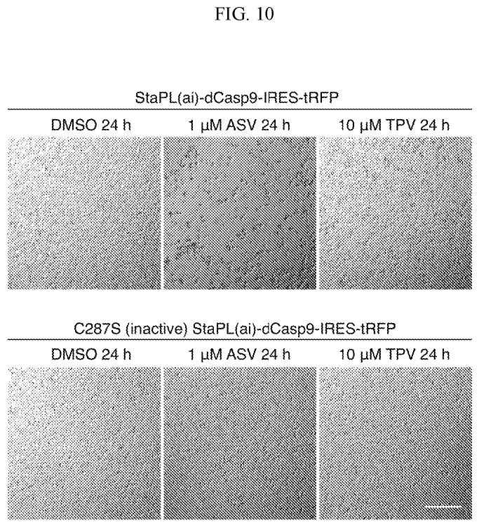

Finally, StaPL sequences were used to enforce protein dimerization through drug-dependent preservation of a tandem dimer. For proteins that are activated by induced homodimerization, this is useful as a method for inducing protein activity with HCV NS3 protease inhibitors. This was tested on Caspase-9, whose activation naturally requires Apaf-1 dependent dimerization of its CARD domain, but which can be activated by fusion to other dimerizing domains (Zhou, X. & Brenner, M. K. Exp Hematol 44, 1013-1019 (2016). A fusion protein comprising, in order, Caspase-9, a StaPL(ai) sequence, and a second copy of Caspase-9, was constructed. As this was in effect a tandem dimer of Caspase-9 regulated by StaPL(ai), it is referred to as StaPL-dCaspase9. Cells expressing StaPL-dCaspase9 survived without drug, but underwent apoptosis in the presence of ASV (FIG. 5, FIG. 10). Thus, StaPL-dCaspase9 effectively functions as a drug-induced active Caspase-9.

In summary, this example describes a system for drug induction of protein activity in which functional protein linkage is selectively retained in the presence of HCV protease inhibitors, a clinically approved class of drugs. As examples, the ability of StaPL sequences to mediate drug preservation of localization tags on proteins and transcriptional regulatory domains on DNA-binding domains was demonstrated. The ability to regulate protein function by insertion of a StaPL sequence in a loop within a domain of dSpCas9, and to regulate local concentration of Caspase-9 domains by using a StaPL sequence to link two copies of Caspase-9 was further demonstrated. A Caspase-9 fusion protein that is dimerized and thereby activated by an experimental drug is being investigated in the clinic as a safety switch to induce apoptosis of transplanted CAR-expressing T cells in case of adverse reactions caused by the cells (Giacca et al., supra). StaPL-dCaspase9 may have similar clinical applications, while using already approved drugs.

StaPL sequences are contemplated for use as drug-stabilized forms of other proteins. As just one example, loop insertion of a StaPL sequence can be performed to create drug-inducible forms of Cas9 proteins from species other than S. pyogenes. When combined with the orthogonal dSpCas9 system, this enables two different genes to be targeted by two different small guide RNAs for their transcription and to be regulated by two different drugs, while involving the expression of only two polypeptide chains. Wildtype Cas9 proteins with intact nuclease activity can also be substituted, in order to carry out drug-activated gene editing. More generally, many proteins tolerate the insertion of protein domains into exposed loops (Heinis, C. & Johnsson, K. Methods Mol Biol 634, 217-232 (2010)). StaPL sequences can be inserted into these loops to create proteins whose folding and thereby function is induced by HCV protease inhibitors.

TABLE 1
NS3 protease mutations yield differential vulnerabilities to protease inhibitor drugs.
Clinically observed HCV genotype 1a NS3 mutations and their effect on the EC50
of ASV or TPV in an in vitro replication assay. V36M, T54A, and S122G increase
vulnerability to ASV and/or resistance to TPV, while F43L, Q80K, S122R, and
D168Y increase vulnerability to TPV and/or resistance to ASV. Data from ref. 12.

| | ASV | Relative | TPV | Relative |
|---|---|---|---|---|
| | EC50 | ASV | EC50 | TPV |
| | (nM) | EC50 | (nM) | EC50 |
| Wt | 0.76 | 1.0 | 181 | 1.0 |
| V36M | 1.5 | 2.0 | 2989 | 16.5 |
| T54A | 0.33 | 0.4 | 847 | 4.7 |
| S122G | 0.65 | 0.9 | 325 | 1.8 |
| F43L | 2.7 | 3.6 | 44 | 0.2 |
| Q80K | 2.5 | 3.3 | 152 | 0.8 |
| S122R | 2.6 | 3.4 | 36 | 0.2 |
| D168Y | 473 | 622 | 58 | 0.3 |

TABLE 2
Comparison of drug dosage dependence between VPR-dCas9(StaPL^{TI}) and PSD95-
SMASh^{TI}. Transcriptional activation of the mCherry reporter by VPR-dCas9(StaPL^{TI}) relies
upon inhibition of the NS3^{TI} protease to preserve dCas9 function, and thus depends on dosage
of TPV inhibitor. Efficient SMASh^{TI} degradation is dependent upon inhibition of the NS3^{TI}
protease, which preserves the linkage between the SMASh degron and PSD95, and is thus
dependent on dosage of TPV inhibitor. mCherry mRNA levels or the efficiency of SMASh in
repressing PSD95 levels were calculated, then each series was scaled to the range of 0 to 1.

| | VPR-dCas9(StaPL^{TI}) | | PSD95-SMASh^{TI} | |
|---|---|---|---|---|
| | mCherry | Scaled | SMASh | Scaled |
| | mRNA | response | efficiency (%) | response |
| DMSO | 1.39 | 0 | 0.00 | 0 |
| 0.1 μM TPV | 2.26 | 0.14 | 11.00 | 0.125 |
| 1 μM TPV | 4.54 | 0.50 | 50.00 | 0.57 |
| 10 μM TPV | 7.70 | 1.00 | 88.00 | 1.00 |

TABLE 3
Comparison of drug dosage dependence between ZF^{VEGFA}-StaPL^{AI}-YFP-VPR and
PSD95-SMASh^{AI}. Transcriptional activation of VEGFA by ZF^{VEGFA}-StaPL^{AI}-YFP-VPR
relies on inhibition of the NS3^{AI} protease preserving the linkage between the ZF DNA
binding domain and the VPR activator, and thus depends on dosage of ASV inhibitor.
Efficient SMASh^{AI} degradation is dependent upon inhibition of the NS3^{AI} protease, which
preserves the linkage between the SMASh degron and PSD95, and is thus dependent on
dosage of ASV inhibitor. VEGFA mRNA levels or the efficiency of SMASh in repressing
PSD95 levels were calculated, then each series was scaled to the range of 0 to 1.

| | ZF^{VEGFA}-StaPL^{AI}-YFP-VPR | | PSD95-SMASh^{AI} | |
|---|---|---|---|---|
| | VEGFA | Scaled | SMASh | Scaled |
| | mRNA | response | efficiency (%) | response |
| DMSO | 1.34 | 0 | 0.00 | 0 |
| 0.01 μM ASV | 1.79 | 0.32 | 28.00 | 0.29 |
| 0.1 μM ASV | 2.48 | 0.80 | 74.00 | 0.77 |
| 1 μM ASV | 2.78 | 1.00 | 96.00 | 1.00 |

### Methods

### DNA Plasmids and Molecular Cloning

Plasmids were constructed using standard molecular biology techniques: restriction enzyme digest (Fermentas), PCR and overlap extension PCR with PrimeSTAR polymerase (Clontech), and assembly using either In-Fusion enzyme (Clontech) or T4 ligase (Thermo Fisher). DNA was transformed into XL10 Gold (Agilent) or Stellar (Clontech) competent E. coli cells with ampicillin (100 μg/mL) selection. Plasmid DNA was isolated with the PureLink hiPure Plasmid Maxiprep Kit (Thermo Fisher). Subcloned regions were verified by Sanger sequencing, assisted by Geneious software (Biomatters).

Hepatitis C Virus sequences used in this study were derived from genotype 1a HCV (Genbank Accession No. NC_004102). Plasmids encoding PSD95-SMASh, Arc-SMASh, and (Venus) YFP-SMASh mutant variants were under the control of the CMV promoter in the pCMV-SPORT6 backbone (Invitrogen), and were adapted from those described in Chung et al., 2015. Plasmids encoding tdYFP-StaPL-NLS and tdRFP-StaPL-NLS, in the pCMV-SPORT6 backbone, consisted of either a tandem pair of Venus YFP connected via a flexible GGSGGS linker (tdYFP) or tdTomato (tdRFP), linked to a StaPL (HCV NS4A-N53) module with an EDVVCC/H cleavage site in between. A tandem repeat of the SV40 nuclear localization sequence (NLS), with sequence DPKKKRKV, was linked C-terminally to the StaPL via a flexible linker.

Zinc finger (ZF) plasmids, in the pCMV-SPORT6 backbone, consisted of a ZF DNA binding domain, connected to a StaPL module via a DEMEEC/S or EDVVCC/H cleavage site, followed by a fluorescent protein marker (either YFP, tdYFP, or tdRFP), a transcriptional effector (either p65, VPR, or p65), and a tandem SV40 NLS attached via a DEMEEC/S cleavage site. The human SP1 protein-derived ZF domain architecture targeted a 10-bp region (GGGGAGGATC) beginning 8 nucleotides upstream of the transcriptional start site (TSS) of the humanVEGFA locus, and was previously described by Liu et al., 2001. The KRAB repressor consisted of the first 97 residues of the human KOX1 protein. The p65 activator consisted of the last 101 residues of the human NF-KB protein. ZF, p65, and KRAB were synthesized de novo by assembly PCR from oligonucleotides, assisted by the DNAWorks algorithm. VPR activator (VP64/p65/Rta) was amplified from a VPR-dSaCas9 plasmid (a gift from the Stanley Qi lab, Stanford) and its internal NLS was replaced with a GGSGGS linker.

The S. pyogenes Cas9 constructs, which carried nuclease-deactivating mutations DlOA and H840A (dSpCas9), were under the control of the PGK promoter, and consisted of an effector domain (either VPR or KRAB) linked to BFP, dSpCas9 (with a DEMEEC/S cleavage site and StaPL module inserted into an internal loop), and a tandem NLS. In the case of VPR, its internal NLS was replaced with a GGSGGSGGS (SEQ ID NO:2) linker. These constructs were adapted from a PGK-VPR-BFP-dSpCas9 plasmid which was obtained from the Stanley Qi lab (Stanford University), as was a U6-sgRNA/CMV-GFP plasmid that expressed a S. pyogenes single-guide RNA directed to the TRE3G locus as well as a GFP marker gene.

Human caspase-9 (excluding its CARD caspase recruitment domain) was amplified from the pET23b-Casp9-His plasmid (from the Guy Salvesen lab, Sanford Burnham Prebys Medical Discovery Institute, Addgene plasmid #11829). Dual copies of the Casp9 large and small subunits were linked together by an HA epitope tag, a DEMEEC/S cleavage site, and a StaPL(ai) module. This StaPL-dCaspase9 construct was subcloned into the pcDNA5/FRT shuttle vector (Thermo Fisher), with a downstream IRES sequence and tRFP (Crimson) marker gene. A catalytic dead variant was also made by mutagenizing both of the large subunits such that they carried a C287S mutation.

### Molecular Modeling

Manipulation of protein crystal structures was performed using UCSF Chimera and MacPyMol. Cocrystals of the HCV NS4A/NS3 protease in complex with asunaprevir (PDB 4WF8) or telaprevir (PDB 3SV6) were modified to include the relevant mutations, and (if applicable) the inhibitor molecule was removed. Structures were then energy minimized in Chimera, and re-imported into MacPyMol for generating images. Modeling of dSpCas9 was performed with a cocrystal of Cas9 with single-guide RNA (PDB 4ZTO).

### Cell Culture and Transfection

HEK293A (Life Tech) and HeLa (ATCC) were passaged in 100 mm dishes (Falcon) and cultured at 37° C. in 5% CO2 in high glucose Dulbecco's modified Eagle's medium (DMEM, Hyclone or Gibco) supplemented with 10% (v/v) fetal bovine serum (FBS, Gemini) or 10% calf serum (Gemini), 2 mM glutamine (Gemini), 100 U/mL penicillin, and 100 μg/mL streptomycin (Gemini) For dSpCas9 experiments, monoclonal stable HEK293 cells with a TRE3G-mCherry cassette were used (obtained from the Stanley Qi lab) and cultured similarly Transfections were performed with Lipofectamine 2000 (Invitrogen) and Opti-MEM (Gibco) according to manufacturer's instructions. Negative control empty vector transfections were performed with pCMV-SPORT6 parent plasmid.

Monoclonal stable HeLa lines expressing StaPL-dCaspase9 constructs were generated via the Flp-In System (Invitrogen). Flp-In T-REx HeLa cells (obtained from the Stephen Taylor lab, University of Manchester) were initially maintained in DMEM supplemented as previously described, and also with 100 μg/mL Zeocin (Thermo Fisher). Transfections occurred in 100 mm dishes in Zeocin-free media. For generating the C287S StaPL(ai)-dCaspase9 cell line, cells were cotransfected with the appropriate pcDNA5/FRT shuttle vector and the Flp recombinase expression vector pOG44 (10% shuttle vector, 90% pOG44, by mass). For the wildtype StaPL(ai)-dCaspase9 cell line, cells were also cotransfected with the pcDNA3-XIAP-Myc plasmid (obtained from the Guy Salvesen lab, Addgene plasmid #11833; 10% shuttle vector, 20% pcDNA3-XIAP-Myc, 70% pOG44, by mass). Coexpression of the antiapoptotic protein XIAP served to rescue cells from premature apoptosis attributable to overexpression of StaPL-dCaspase9. After 48 hours of expression, cells were trypsinized with 0.25% Trypsin EDTA solution (Gemini) and replated in 12 well plates (Greiner) in DMEM containing 200 μg/mL Hygromycin B (Gibco) for selection. Transformant colonies were picked, trypsinized, expanded, and verified by vulnerability to zeomycin, tRFP fluorescence, HA immunopositivity by western blot, and by extracting genomic DNA (DNeasy Blood and Tissue Kit, Qiagen) for PCR amplification and sequencing confirmation of the inserted gene. Downstream experiments were performed in Hygromycin B-containing DMEM.

### Chemical Reagents

Telaprevir (TPV; VX-950) was obtained from AdooQ Biosciences and asunaprevir (ASV; BMS-650032) was obtained by custom synthesis (Acme Bioscience). (ASV can also be purchased from AdooQ Bioscences or Santa Cruz Biotechnology). Dimethyl sulphoxide (DMSO, Santa Cruz Biotechnology) stocks were made at 1000× target concentration and stored at −20° C. TPV and ASV were applied to cells directly prior to transfection, or along with transfection reagents. Cobalt(II) chloride hexahydrate (CoCl₂, Sigma Aldrich) was dissolved in water to make a 400 mM (500×) stock. In applicable experiments, cells were incubated in 800 μM CoCl2 for the final 7 hrs before harvest, in order to simulate hypoxia.

### Immunoblotting

For SDS-PAGE analysis, cells were cultured in 24 or 12 well plates, and lysed 8-48 hrs after transfection with 50 or 100 μl of hot (90° C.) SDS lysis buffer (100 mM Tris HCl pH 8.0, 3% SDS, 20% glycerol, 0.2% bromophenol blue, 10% 2-mercaptoethanol). Lysates were sonicated to shear DNA, heated briefly to 90° C., and centrifuged prior to loading on either NuPAGE 4-12% Bis-Tris (Invitrogen) or 4-15% Criterion TGX (Bio-Rad) gels, along with Novex Sharp pre-stained protein standard (Life Tech). Transfers onto PVDF membrane were performed using either the iBlot system (Life Tech) or the Trans-Blot Turbo system (Bio-Rad).

Membranes were typically probed with primary and secondary antibodies using the iBind sytem (Life Tech). Alternatively, immunoprobing was done by blocking the membrane with 7.5% (w/v) nonfat dry milk in Tris-buffered saline (TBS) for 1 hr at ambient temperature on an electric rocker, washing 3× using TBS with 0.1% Tween 20 (TBS-T), incubating 1 hr with primary antibodies in 5% bovine serum albumin (BSA) in TBS-T, washing 3× in TBS-T, incubating 1 hr with fluorescent secondary antibodies in 7.5% nonfat dry milk in TBS-T, and again washing 3× in TBS-T. In some instances, membranes were cut in order to stain two sections separately with different antibodies. Membranes were scanned using a LI-COR Odyssey or CLx imager.

Where applicable, western blot quantifications were performed using ImageJ and Fiji. Integrated densities for bands of the same protein species were measured using a consistently sized rectangle, and background measurements from the same lane were subtracted from these values. Protein of interest bands were normalized via dividing by loading control bands, which were quantified by the same method. Data analysis was performed using Microsoft Excel and Apache OpenOffice software.

### Antibodies

The following primary antibodies were used for immunoblotting at the indicated dilutions: mouse monoclonal anti-PSD95 (NeuroMab, clone K28/43), 1:1000; rabbit polyclonal anti-β-actin (GeneTex, GTX124214), 1:3333; rabbit polyclonal anti-GAPDH (Santa Cruz, FL-335/sc-25778), 1:500; rabbit polyclonal anti-Arc (Synaptic Systems, #156 003), 1:200; mouse monoclonal anti-GFP (Pierce, clone GF28R/MA5-15256), 1:1000; rabbit polyclonal anti-tdTomato (OriGene, TA150128), 1:2000; rabbit monoclonal anti-HA (Cell Signaling, C29F4), 1:1000; rabbit polyclonal anti-tRFP (Evrogen, AB233), 1:1000; mouse monoclonal anti-β-actin (Santa Cruz, ACTBD11B7/sc-81178), 1:1000. Secondary antibodies used were either LI-COR 680RD goat-anti-mouse and 800CW goat-anti-rabbit, or 680RD goat-anti-rabbit and 800CW goat-anti-mouse. Secondaries were used at 1:3333.

### Microscopy

Brightfield microscopy of live stable StaPL-dCaspase9 HeLa cells (in 12 well plates, Greiner) was performed on an EVOS FL Cell Imaging System with a 4×0.1NA air objective. Fluorescence widefield microscopy of live transfected HeLa cells (in 35 mm glass bottom 4-chamber dishes, In Vitro Scientific), fixed transfected TRE3G-mCherry stable HEK293 cells (in glass bottom 12 well plates, In Vitro Scientific), and stained/fixed stable StaPL-dCaspase9 HeLa cells (mounted on glass slides with #1.5 cover glass) was performed on a Zeiss Axiovert 200M inverted microscope with a 40×1.2NA water immersion, 5×0.25NA air, or 10×0.5NA air objective, respectively. This microscope was equipped with an X-Cite 120-W metal-halide lamp and a 3 mm core liquid light guide (Lumen Dynamics), connected to a Hamamatsu ORCA-ER camera, and controlled with Micro-Manager software. The following excitation (ex) and emission (em) filters were used: Blue (DAPI), ex 380/14 nm (Semrock), em 420 nm longpass dichroic (Olympus) and 442/46 nm (Semrock); Green/Yellow (YFP, GFP, Alexa 488), ex 485/10 nm (Chroma), em 510 nm longpass dichroic (Omega) and 525/40 nm (Chroma); Red (RFP, tRFP), ex 568/20 nm (Omega), em 585 longpass dichroic (Chroma) and 620/60 nm (Chroma).

Confocal fluorescence microscopy of live and fixed transfected HEK293A cells (in 35 mm glass bottom 4-chamber dishes, In Vitro Scientific) was performed on a PerkinElmer UltraVIEW VoX system equipped with a CSU-X1 spinning disc (Yokogawa Electric), a Hamamatsu EM CCD C9100-50 camera, and controlled by Volocity 5 software (Improvision). Imaging was performed with an a-plan Apochromat 63×1.4NA oil immersion objective (Carl Zeiss). The following excitation (ex) lasers (at 30% power) and emission (em) filters were used: Green/Yellow (YFP), ex 488 nm laser, em 525/50 nm; Red (RFP), ex 561 nm laser, em 615/70 nm.

Glass bottom cell culture vessels for HEK293/HEK293A cells were typically coated with 0.5 mg/mL poly-D-lysine hydrobromide (Sigma-Aldrich) in sterile water by incubating at 37° C. overnight, and were washed 4x with water prior to plating cells. Live StaPL-dCaspase9 HeLa cells were imaged in DMEM, and live transfected HeLa cells were imaged in HBSS. Live transfected HEK293A cells were imaged in FluoroBrite DMEM (Gibco) supplemented as previously described, and with appropriate protease inhibitor(s), in a light-protected chamber maintained at 33° C. Fixed cells were fixed using 4% paraformaldehyde (PFA, Electron Microscopy Sciences) in phospho-buffered saline (PBS, HyClone) for 15 min at ambient temperature, washed 2× with Hank's Buffered Salt Solution (HBSS, HyClone), and kept in HBSS subsequently.

For stained/fixed stable StaPL-dCaspase9 HeLa cells, 10 μm z-stacks were acquired in 1 μm intervals and were subsequently transformed into maximum intensity projections. For live and fixed transfected HEK293A cells, z-stacks were acquired in 0.5 μm intervals and ranges encompassing 4 μm were transformed into maximum intensity projections. Image processing and analysis was performed with ImageJ and Fiji.

### Enzyme-Linked Immunosorbent Assay (ELISA)

HEK293A cells were cultured in 12 well plates (Greiner) with 1 mL of DMEM (supplemented as previously described) in each well, and were transfected 48 hrs prior to harvesting media supernatants (transfection reagent volume, 200 μl). Media were not changed after transfection. Appropriate protease inhibitor drugs were applied concurrent with transfection. Cells transfected with empty vector and given identical protease inhibitor treatments served as controls. From each well, 900 μl of media supernatant was collected for ELISA analyte, and frozen at −20° C. until it was processed. Samples were thawed to ambient temperature one hour before use, and cleared by centrifuging at 10,000×g for 10 min at 4° C.

Sandwich ELISA was performed using the Human VEGF Quantikine ELISA Kit (R&D Systems) according to manufacturer's protocol. Absorbances at 450 nm (with reference wavelength 450 nm) were measured thrice and averaged, using a Tecan Infinite M1000 PRO plate reader under the control of Tecan i-control software Human VEGF protein dilution standards were used to calculate sample [VEGF] values by 4-parameter logistic regression (www.elisaanalysis.com). In some cases, sample measurements exceeded those of the top standard value, and were diluted twofold and rescanned in order to interpolate them. The calculated [VEGF] for such samples was thus multiplied by two. Since each well of HEK293A contained 1.2 mL of media post-transfection, [VEGF] values were adjusted by factor 1.2 in order to obtain true concentrations in pg/mL. VEGF measurements are expressed as differences from the appropriate empty vector control value (from cells incubated in alike protease inhibitor conditions).

### Cell Staining

For staining of StaPL-dCaspase9 HeLa cells, media were collected to harvest dead/lifted cells, adherent cells were trypsinized to harvest living cells, and the two were pooled and centrifuged at 500×g for 5 min to pellet them. Cells were washed once with HBSS, resuspended in either HBSS or Annexin V Binding Buffer (Biotium), and stained with the NucView 488 Caspase-3 Assay Kit for Live Cells (Biotium) or the Annexin V CF488A Conjugate (Biotium), respectively, according to manufacturer's instructions. For annexin V staining, cells of the parent cell line (Flp-In HeLa) were stained in parallel. For medium-term preservation, cells were fixed in 4% PFA in PBS for 15 min at ambient temperature following staining, centrifuged at 10,000×g, and resuspended in HBSS after aspirating the PFA. A 25 μl droplet of each sample was then placed on a Superfrost Plus glass slide (Fisher Scientific), and allowed to dry partially, before adding VECTASHIELD Mounting Medium with DAPI (Vector Labs) and a #1.5 cover glass (Fisher Scientific). Slides were sealed with clear nail polish and kept at 4° C.

### Flow Cytometry

After 24 hrs of drug incubation, StaPL-dCaspase9 HeLa cells and cells of the parent cell line (Flp-In HeLa) were harvested and stained. Flow cytometry was performed on live and fixed stained cells using a Digital Vantage instrument (Becton Dickinson) under the control of CellQuest software (Becton Dickinson), operated by the Stanford Shared FACS Facility. A 488 nm laser was used to excite in Green (Alexa 488 stain) and a 594 nm laser was used to excite in Red (Crimson tRFP). The parent Flp-In HeLa cell line, which did not express tRFP, assisted in defining the tRFP-positive population. For each sample, 10,000 events were collected. Cytometry data was analyzed and processed using FlowJo software.

All publications and patents mentioned in the above specification are herein incorporated by reference as if expressly set forth herein. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in relevant fields are intended to be within the scope of the following claims.

1. A composition, comprising:
a fusion protein comprising a) a first polypeptide of interest; and b) a first protease and a substrate for said protease.

2. The composition of claim 1, wherein said protease and said substrate are inserted between multiple domains of said polypeptide.

3. The composition of claim 1, wherein said protease and said substrate are inserted within a domain of said polypeptide of interest.

4. The composition of claim 1, wherein said protease and said substrate are inserted between two copies of said polypeptide of interest.

5. The composition of any one of claims 1 to 4, wherein said composition comprises a second fusion protein comprising second polypeptide of interest and a second protease, wherein said second protease is distinct from said first protease.

6. The composition of claim 5, wherein said first protease and said second protease are inhibited by different protease inhibitors.

7. The composition of any one of claims 1 to 6, wherein said first and second proteases are HCV NS3 proteases.

8. The composition of claim 7, wherein said HCV NS3 proteases comprise at least one mutation.

9. The composition of claim 8, wherein said first HCV NS3 protease comprises V36M, T54A, and S122G mutations and said second HCV NS3 protease comprises F43L, Q80K, S122R, and D168Y mutations.

10. The composition of claim 9, wherein said HCV NS3 protease comprising V36M, T54A, and S122G mutations is resistant to telaprevir (TPV) and sensitive to asunaprevir (ASV) and said HCV NS3 protease comprising F43L, Q80K, S122R, and D168Y mutations is resistant to ASV and sensitive to TPV.

11. A nucleic acid encoding the fusion protein of any one of claims 1 to 10.

12. A cell comprising the nucleic acid of claim 11 or the fusion protein of any one of claims 1 to 10.

13. The cell of claim 12, wherein said nucleic acid is on a chromosome of said cell.

14. The cell of claim 12 or 13, wherein said cell is in an organism.

15. The cell of claim 14, wherein said organism is selected from the group consisting of a microorganism, a non-human animal, and a human.

16. The composition of any one of claims 1 to 10, wherein said protein of interest is selected from the group consisting of a transcription factor, a nuclease enzyme, a protease enzyme, and a metabolic enzyme.

17. A kit or system, comprising:
a) the nucleic acid of claim 11; and
b) at least one protease inhibitor.

18. A method of modulating the activity or function of a polypeptide of interest, comprising:
contacting the cell of any one of claims 12 to 15 with a protease inhibitor under conditions such that said polypeptide of interest is active.

19. The method of claim 18, wherein said polypeptide of interest is selected from the group consisting of a transcription factor, a nuclease enzyme, a protease enzyme, and a metabolic enzyme.


SEQUENCE LISTING (3 sequences)

SEQ ID NO: 1 - protein, 193 aa, Artificial sequence
  synthetic
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAV

SEQ ID NO: 2 - protein, 9 aa, Artificial sequence
  synthetic
GGSGGSGGS

SEQ ID NO: 3 - protein, 220 aa, Artificial sequence
  synthetic
TGCVVIVGRIVLSGSGTSAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLPRPISYLKGSSGGPLLCPAGHAVGLFRAAVCTRGVAKAVDFIPVENLETTMRSPVFTDNSSPPAVTLTHGGSGGS